SEQUENCE LISTING (1 sequence)

SEQ ID NO: 1 - DNA, 30 bp, synthetic construct
  source 1..30: mol_type = other DNA; note = Description of Artificial Sequence: Synthetic oligonucleotide
  modified_base 1: mod_base = OTHER; note = thymidine phosphorothioate
  modified_base 2: mod_base = OTHER; note = cytidine phosphorothioate
  modified_base 3: mod_base = OTHER; note = guanosine phosphorothioate
  modified_base 4: mod_base = OTHER; note = adenosine phosphorothioate
  modified_base 5: mod_base = OTHER; note = adenosine phosphorothioate
  modified_base 6: mod_base = OTHER; note = cytidine phosphorothioate
  modified_base 7: mod_base = OTHER; note = guanosine phosphorothioate
  modified_base 8: mod_base = OTHER; note = thymidine phosphorothioate
  modified_base 9: mod_base = OTHER; note = thymidine phosphorothioate
  modified_base 10: mod_base = OTHER; note = cytidine phosphorothioate
  modified_base 11: mod_base = OTHER; note = guanosine phosphorothioate
  modified_base 12: mod_base = OTHER; note = adenosine phosphorothioate
  modified_base 13: mod_base = OTHER; note = adenosine phosphorothioate
  modified_base 14: mod_base = OTHER; note = cytidine phosphorothioate
  modified_base 15: mod_base = OTHER; note = guanosine phosphorothioate
  modified_base 16: mod_base = OTHER; note = thymidine phosphorothioate
  modified_base 17: mod_base = OTHER; note = thymidine phosphorothioate
  modified_base 18: mod_base = OTHER; note = cytidine phosphorothioate
  modified_base 19: mod_base = OTHER; note = guanosine phosphorothioate
  modified_base 20: mod_base = OTHER; note = adenosine phosphorothioate
  modified_base 21: mod_base = OTHER; note = adenosine phosphorothioate
  modified_base 22: mod_base = OTHER; note = cytidine phosphorothioate
  modified_base 23: mod_base = OTHER; note = guanosine phosphorothioate
  modified_base 24: mod_base = OTHER; note = thymidine phosphorothioate
  modified_base 25: mod_base = OTHER; note = thymidine phosphorothioate
  modified_base 26: mod_base = OTHER; note = cytidine phosphorothioate
  modified_base 27: mod_base = OTHER; note = guanosine phosphorothioate
  modified_base 28: mod_base = OTHER; note = adenosine phosphorothioate
  modified_base 29: mod_base = OTHER; note = adenosine phosphorothioate
tcgaacgttcgaacgttcgaacgttcgaat

(19) United States
(12) Patent Application Publication
(10) Pub. No.: US 2023/0193374 A1
(43) Pub. Date: Jun. 22, 2023

(54) TISSUE HOMOGENIZATION BUFFER FOR EFFICIENT QUANTITATION OF A THERAPEUTIC OLIGONUCLEOTIDE IN MULTIPLE TISSUES

(71) Applicant: TriSalus Life Sciences, Inc., Westminster, CO (US)

(72) Inventors: Hongqiao Lin, Colorado Springs, CO (US); Edward Brewer, Colorado Springs, CO (US); David Benjamin Jaroch, Arvada, CO (US)

(73) Assignee: TriSalus Life Sciences, Inc., Westminster, CO (US)

(21) Appl. No.: 17/951,874

(22) Filed: Sep. 23, 2022

Related U.S. Application Data
(60) Provisional application No. 63/248,205, filed on Sep. 24, 2021.

(51) Int. Cl. C12Q 1/6869; G01N 1/34

(52) U.S. Cl. CPC C12Q 1/6869; G01N 1/34; C12Q 1/6806

(57) ABSTRACT

Embodiments of the present invention provide for an optimized tissue homogenization buffer composition and a method of using an optimized tissue homogenization buffer for efficient quantitation of a therapeutic oligonucleotide in multiple tissues. The exemplary method comprising: mixing the tissue with a homogenization buffer composition to create a tissue homogenate; adding the tissue homogenate to a quantity of oligonucleotide-free plasma to create a tissue homogenate solution; adding a quantity of a reference standard oligonucleotide to the tissue homogenate solution to create a homogenate/plasma/standard solution; adding a quantity of a phenol/chloroform/isoamyl alcohol to the homogenate/plasma/standard solution; employing centrifugal force to separate a supernatant from the homogenate/plasma/standard solution; and analyzing the supernatant for a concentration of the oligonucleotide.

### CROSS REFERENCE TO RELATED APPLICATION(S)

This application claims the benefit of U.S. Provisional Patent Application No. 63/248,205, which was filed on Sep. 24, 2021, which is incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 16, 2020, is named A372-502_SL.txt and is 484 bytes in size.

### FIELD OF THE INVENTION

The present disclosure relates generally to a method for processing tissues for oligonucleotide and tissue distribution assessment.

### BACKGROUND OF THE INVENTION

Synthetic oligonucleotides have gained popularity as novel therapeutics for disease treatment which led to increasing interest in measurement of therapeutic oligonucleotides in biological matrices to provide essential information for drug development such as pharmacokinetic (PK) evaluation, toxicokinetic (TK) evaluation, and tissue distribution.

For early drug development work, there is a need to develop one assay that can be used for the evaluation of multiple biological matrices. In small molecule drug development, the practice of utilizing a plasma assay to analyze tissue homogenates has been well established. However, adequate buffer chemistries for processing multiple tissue types for oligonucleotide PK/TK and tissue distribution assessment has not been reported.

Accordingly, it would be desirable to have solutions that overcome these and other deficiencies.

### SUMMARY OF THE INVENTION

The present invention relates to a buffer composition and method of tissue homogenization applicable to multiple tissue matrix types present within a body for the quantification of oligonucleotide therapeutics.

In one aspect, the present invention relates to utilizing a plasma assay to analyze an oligonucleotide in various tissue matrices.

In one aspect, the present invention relates to an exemplary buffer composition containing an optimized pH and organic/aqueous ratio which eliminates the difference in matrix effect between the plasma calibration standard and tissue quality control (QC)/sample matrices.

In one aspect, the present invention relates to an exemplary homogenization buffer composition comprising: ammonium acetate in solution in water, wherein a concentration of the ammonium acetate within the homogenization buffer composition is about 5 mM to 20 mM; and a quantity of proteinase K, wherein the quantity of proteinase K is from 50 μg to 200 μg for every 1 mL of the homogenization buffer composition; wherein: (i) the pH of the homogenization buffer composition is between 3.0 to 6.0 pH; and (ii) the homogenization buffer composition is in a concentration ranging from 4 mL of the homogenization buffer composition per 1 gram of the tissue to 200 mL of the homogenization buffer composition per 1 gram of the tissue.

In one aspect, the present invention relates to a method for quantitation of an oligonucleotide within a tissue including: mixing the tissue with a homogenization buffer composition to create a tissue homogenate, wherein the homogenization buffer composition comprises ammonium acetate in solution in water; adding the tissue homogenate to a quantity of oligonucleotide-free plasma to create a tissue homogenate solution; adding a quantity of a reference standard oligonucleotide to the tissue homogenate solution to create a homogenate/plasma/standard solution; adding a quantity of a phenol/chloroform/isoamyl alcohol to the homogenate/plasma/standard solution; employing centrifugal force to separate a supernatant from the homogenate/plasma/standard solution; and analyzing the supernatant for a concentration of the oligonucleotide, wherein the oligonucleotide is a toll-like receptor 9 agonist oligonucleotide having the structure: 5′-TCG AAC GTT CGA ACG TTC GAA CGT TCG AAT-3′ (SEQ ID NO: 1).

In some embodiments, the present invention relates to an exemplary method to quantitate an oligonucleotide in six different porcine tissue matrices against plasma calibration curve. In this regard, the exemplary method can use 10 mM ammonium acetate (which has a pH of 4.5) as the tissue homogenization buffer. Distinct from the traditional tissue assays, tissue QC samples can be matrix-matched with plasma during extraction; while the plasma calibration curve and QC samples can be matched with the homogenization buffer only. Both the plasma and tissue QC samples show satisfactory accuracy and precision. This can be demonstrated by using a buffer with optimized pH and an organic/aqueous ratio, which can successfully eliminate the difference in matrix effect between the plasma calibration standard and the tissue QC/sample matrices. Further, this approach of analysis of oligonucleotides in tissue provides a fast and easy way to analyze multiple different tissue samples together and improves the efficiency in determining the oligonucleotide drug tissue distribution studies for the drug discovery program.

In some embodiments, the buffer contains ammonium acetate in water.

In some embodiments, the buffer contains ammonium acetate in water and methanol.

In some embodiments, the buffer contains ammonium acetate in solution having a pH of 4.45.

In some embodiments, the buffer contains proteinase K.

In some embodiments, the tissue is porcine in origin.

In some embodiments, the tissue is human in origin.

In some embodiments, the tissue is from at least one of the liver, lung, brain, spleen, thymus, and lymphoid tissue.

In some embodiments, the tissue is normal tissue and does not include any cancerous tumors.

In some embodiments, the buffer is mixed with the tissue in a ratio of 9 mL of solvent to 1 gram of tissue.

In some embodiments, the buffer-to-tissue ratio is in a range from 20:1 to 100:1.

In some embodiments, the buffer-to-tissue ratio is 50:1.

In some embodiments, the oligonucleotide is a TLR agonist.

In some embodiments, the TLR agonist is a TLR9 agonist.

In some embodiments, the oligonucleotide is a Class C type CpG oligodeoxynucleotide (CpG-C ODN).

In some embodiments, the CpG-C ODN can comprise SD-101.

These and other objects, features, and advantages of the exemplary embodiments of the present disclosure will become apparent upon reading the following detailed description of the exemplary embodiments of the present disclosure, when taken in conjunction with the entire specification.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure.

FIG. 1 illustrates the structure of SD-101.

FIG. 2 illustrates an exemplary set of parameters and solutions used for sample analysis by high performance liquid chromatography tandem mass spectroscopy (HPLC-MS/MS).

FIG. 3 illustrates a representative porcine plasma calibration curve in tissue qualification.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION

The following description of embodiments provides non-limiting representative examples referencing numerals to particularly describe features and teachings of different aspects of the invention. The embodiments described should be recognized as capable of implementation separately, or in combination, with other embodiments from the description of the embodiments. A person of ordinary skill in the art reviewing the description of embodiments should be able to understand the different described aspects of the invention. The description of embodiments should facilitate understanding of the invention to such an extent that other implementations, not specifically covered but within the knowledge of a person of skill in the art having read the description of embodiments, would be understood to be consistent with application of the invention.

### Toll-like Receptor Agonists

Toll-like receptors are pattern recognition receptors that can detect microbial pathogen-associated molecular patterns (PAMPs). TLR stimulation, such as TLR9 stimulation, can not only provide broad innate immune stimulation, but can also specifically address the dominant drivers of immunosuppression in the liver. TLR1-10 are expressed in humans and recognize a diverse variety of microbial PAMPs. In this regard, TLR9 can respond to unmethylated CpG-DNA, including microbial DNA. CpG refers to the motif of a cytosine and guanine dinucleotide 1. TLR9 is constitutively expressed in B cells, plasmacytoid dendritic cells (pDCs), activated neutrophils, monocytes/macrophages, T cells, and MDSCs. TLR9 is also expressed in non-immune cells, including keratinocytes and gut, cervical, and respiratory epithelial cells. TLR9 can bind to its agonists within endosomes. Signaling may be carried out through MYD88/IkB/NfκB to induce pro-inflammatory cytokine gene expression. A parallel signaling pathway through IRF7 induces type 1 and 2 interferons (e.g., IFN-α, IFN-γ, etc.) which stimulate adaptive immune responses. Further, TLR9 agonists can induce cytokine and IFN production and functional maturation of antigen presenting dendritic cells.

According to an embodiment, synthetic CpG-oligonucleotides (CPG-ONs) mimicking the immunostimulatory nature of microbial CpG-DNA can be developed for therapeutic use. According to an embodiment, the oligonucleotide is an oligodeoxynucleotide (ODN). There are a number of different CpG-ODN class types, e.g., Class A, Class B, Class C, Class P, and Class S, which share certain structural and functional features. In this regard, Class A type CPG-ODNs (or CPG-A ODNs) are associated with pDC maturation with little effect on B cells as well as the highest degree of IFNα induction; Class B type CPG-ODNs (or CPG-B ODNs) strongly induce B-cell proliferation, activate pDC and monocyte maturation, NK cell activation, and inflammatory cytokine production; and Class C type CPG-ODNs (or CPG-C ODNs) can induce B-cell proliferation and IFN-α production. Further, according to an embodiment, CPG-C ODNs can be associated with the following attributes: (i) unmethylated dinucleotide CpG motifs, (ii) juxtaposed CpG motifs with flanking nucleotides (e.g., AACGTTCGAA), (iii) a complete phosphorothioate (PS) backbone that links the nucleotides (as opposed to the natural phosphodiester (PO) backbones found in bacterial DNA), and (iv) a self-complimentary, palindromic sequence (e.g., AACGTT). In this regard, CPG-C ODNs may bind themselves due to their palindromic nature, thereby producing double-stranded duplex or hairpin structures.

Further, according to an embodiment, the CPG-C ODNs can include one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In an embodiment, the CpG-C ODNs are 12 to 100 bases in length, preferably 12 to 50 bases in length, preferably 12 to 40 bases in length, or preferably 12-30 bases in length. In an embodiment, the CpG-C ODN is 30 bases in length. In an embodiment, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In an embodiment, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length.

In an embodiment, the at least one palindromic sequence is 8 to 97 bases in length, preferably 8 to 50 bases in length, or preferably 8 to 32 bases in length. In an embodiment, the at least one palindromic sequence is at least (lower limit) 8, 10, 12, 14, 16, 18, 20, 22, 24, 26, 28, or 30 bases in length. In an embodiment, the at least one palindromic sequence is at most (upper limit) 50, 48, 46, 44, 42, 40, 38, 36, 34, 32, 30, 28, 26, 24, 22, 20, 18, 16, 14, 12 or 10 bases in length.

In an embodiment, the CpG-C ODN can comprise the sequence of SEQ ID NO: 1.

According to an embodiment, the CpG-C ODN can comprise the SD-101. SD-101 is a 30-mer phosphorothioate oligodeoxynucleotide, having the following sequence:

(SEQ ID NO: 1)
5′-TCG AAC GTT CGA ACG TTC GAA CGT TCG AAT-3′

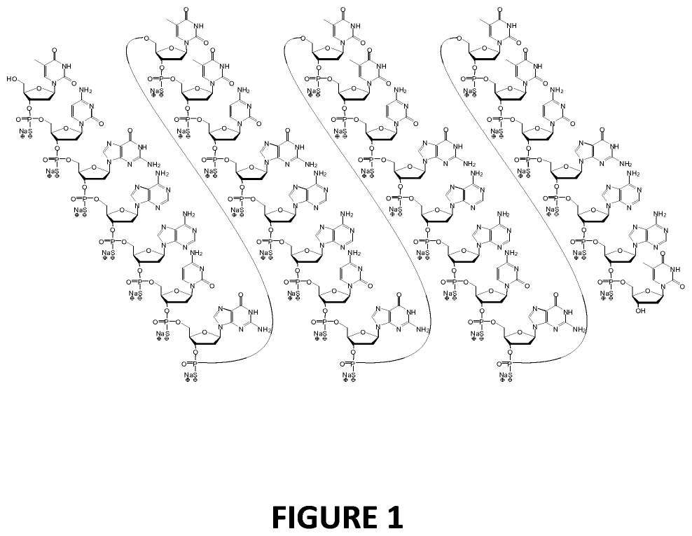

SD-101 drug substance is isolated as the sodium salt. The structure of SD-101 is illustrated in FIG. 1. The molecular formula of SD-101 free acid is C₂₉₃H₃₆₉N₁₁₂O₁₄₉P₂₉S₂₉ and the molecular mass of the SD-101 free acid is 9.672 Daltons. The molecular formula of SD-101 sodium salt is C₂₉₃H₃₄₀N₁₁₂O₁₄₉P₂₉S₂₉Na₂₉ and the molecular mass of the SD-101 sodium salt is 10,309 Daltons.

Further, according to an embodiment, the CPG-C ODN sequence can correspond to SEQ ID NO:172 as described in U.S. Pat. No. 9,422,564, which is incorporated by reference herein in its entirety.

In an embodiment, the CpG-C ODN can comprise a sequence that has at least 75% homology to any of the foregoing, such as SEQ ID NO:1.

According to another embodiment the CPG-C ODN sequence can correspond to any one of the other sequences described in U.S. Pat. No. 9,422,564. Further, the CPG-C ODN sequence can also correspond to any of the sequences described in U.S. Pat. No. 8,372,413, which is also incorporated by reference herein in its entirety.

### Buffer Solution for Tissue Homogenization

In some embodiments, the buffer solution is comprised of at least ammonium acetate in solution in water. The concentration of ammonium acetate may range from 5 mM to 20 mM. In an exemplary embodiment, the concentration of ammonium acetate is 10 mM in water.

In an embodiment the aqueous solution may contain an organic solvent. In an embodiment the organic solvent is methanol. In an embodiment the weight ratio of water to solvent ranges from 10/90 methanol/water to 90/10 methanol/water. In an embodiment the weight ratio of methanol to water is 50/50. In an embodiment the weight ratio of methanol to water is 20/80.

In an embodiment of the invention, the buffer solution pH is adjusted between 6.0 to 3.0 pH. In an embodiment, the buffer solution pH is adjusted to 4.5 pH.

In some embodiments, the buffer contains 50 μg to 200 μg of proteinase K for every 1 mL of buffer.

In some embodiments, the buffer contains 50 μg of proteinase K for every 1 mL of buffer.

Methods Comprising Tissue Homogenization

In an embodiment, the methods of the present invention include the use of a buffer as previously described to homogenize a porcine or human tissue from at least one of the liver, lung, brain, spleen, thymus and lymphoid tissue.

In an embodiment, the tissue is mixed with the buffer in a range of 4 mL of buffer per 1 gram of tissue to 200 mL of buffer per 1 gram of tissue.

In an embodiment, the tissue is mixed with the buffer in a range of 20 mL of buffer per 1 gram of tissue to 100 mL of buffer per 1 gram of tissue. In an embodiment, the tissue is mixed with the buffer in a range of 20 mL of buffer per 1 gram of tissue to 50 mL of buffer per 1 gram of tissue.

In an embodiment, the tissue is mixed with the buffer in a ratio of 50 mL of buffer per 1 gram of tissue.

In an embodiment, the tissue is mixed with the buffer in a range of 19 mL of buffer per 1 gram of tissue to 4 mL of buffer per 1 gram of tissue.

In an embodiment, the tissue is mixed with the buffer in a ratio of 9 mL of buffer per 1 gram of tissue.

In an embodiment, ceramic beads may be added to the solution.

In an embodiment, the solution with ceramic beads is mechanically agitated until the tissue is fully dissociated.

In an embodiment, the tissue is frozen and pulverized, e.g., with ceramic beads, until fully dissociated prior to mixing with buffer.

### Methods for Sample Extraction

In an embodiment, a method for quantitation of an oligonucleotide within a tissue can include: mixing the tissue with a homogenization buffer composition to create a tissue homogenate, wherein the homogenization buffer composition comprises ammonium acetate in solution in water; adding the tissue homogenate to a quantity of oligonucleotide-free plasma to create a tissue homogenate solution; adding a quantity of a reference standard oligonucleotide to the tissue homogenate solution to create a homogenate/plasma/standard solution; adding a quantity of a phenol/chloroform/isoamyl alcohol to the homogenate/plasma/standard solution; employing centrifugal force to separate a supernatant from the homogenate/plasma/standard solution; and analyzing the supernatant for a concentration of the oligonucleotide, wherein the oligonucleotide is a toll-like receptor 9 agonist oligonucleotide having the structure: 5′-TCG AAC GTT CGA ACG TTC GAA CGT TCG AAT-3′ (SEQ ID NO: 1).

In an embodiment, samples are extracted by addition of at least tissue homogenate to ODN free porcine or human plasma and internal standard ODN.

In an embodiment, the ratio of tissue homogenate to ODN free plasma is 50:50 by volume.

In an embodiment, the proportions of the solution are 200 μL of ODN free plasma, 200 μL tissue homogenate, and 20.0 μL of the 1000-ng/mL internal standard ODN.

In an embodiment, 100 μL of the 25/24/1 phenol/chloroform/isoamyl alcohol is added to homogenate/plasma/standard solution.

In an embodiment, centrifugal force is employed to separate supernatant for LC-MS/MS analysis.

In an embodiment, SD-101 can be extracted from normal tissue near tumor at a buffer-to-tissue ratio range of 4:1 to 200:1, a buffer-to-tissue ratio range of 20:1 to 100:1, or a buffer-to-tissue ratio of 50:1.

In an embodiment, the inclusion of proteinase K in the buffer composition facilitates extraction of SD-101 from normal tissue. In an embodiment, the buffer composition contains 50 μg to 200 μg of proteinase K for every 1 mL of buffer composition. In an embodiment, the buffer composition contains 50 μg of proteinase K for every 1 mL of buffer composition.

### Example 1

The present example describes a bioanalytical method to determine quantification of SD-101, a 30-base synthetic phosphorothioate oligonucleotide in various porcine tissue matrices with phenol/chloroform liquid-liquid extraction and ion-pairing reversed-phase HPLC coupled with negative ion ESI-MS/MS detection.

Six different buffers were tested to homogenize the porcine tissues. The buffers are:
1) 10 mM Tris in water, with a pH of 8.0;
2) 10 mM Tris in 20/80 methanol/water, with a pH of 8.0;
3) 10 mM Tris in 50/50 methanol/water, with a pH of 8.0;
4) 10 mM ammonium acetate in water, with a pH of 4.5;
5) 10 mM ammonium acetate in 20/80 methanol/water, with a pH of 4.5; and
6) 10 mM ammonium acetate in 50/50 methanol/water, with a pH of 4.5.

Through optimization of tissue homogenization buffer, one method was used to analyze six different porcine tissues with a porcine plasma calibration curve.

Multiple porcine tissues including liver, lung, brain, spleen, thymus and lymphoid tissue, which were previously frozen and pulverized (e.g., with ceramic beads), were thawed and weighed. The homogenizations were performed by adding appropriate volume of homogenization buffer (see 1) to 6) above) to each sample using a 9:1 ratio of solvent to tissue (9 mL of solvent to 1 gram of tissue), resulting in a 10× dilution. Ceramic beads were added to each tube and the tissue samples were vortex-mixed (homogenized) twice using a Geno Grinder for 2 minutes at 1700 rpm.

The range of quantitation analysis for SD-101 in tissue samples was from the Lower Limit of Quantification (LLOQ), 10.0 ng/g, to the Upper Limit of Quantification (ULOQ), 5000 ng/g. QC recovery samples were prepared in porcine plasma and various control tissue homogenate at 3 ng/mL, 15 ng/mL, 150 ng/mL and 350 ng/mL, which corresponds to 30.0 ng/g, 150 ng/g, 1500 ng/g, and 3500 ng/g, respectively, in tissue equivalence. Fortification solutions were prepared by serial dilution of the stock solution in water. The calibration standards and QC samples were prepared fresh for each run by spiking the correspondent fortification solution to porcine K₂EDTA plasma and porcine tissue homogenate, mixed by vortex following preparation, and mixed again prior to use.

Sample extraction was then performed. 200 μL of porcine plasma calibration standards and plasma or tissue QC samples were each extracted in 1.5 mL Eppendorf tubes by adding 200 μL of appropriate homogenization buffer to each plasma sample and adding 200 of blank porcine plasma (K₂EDTA) to the tissue sample. 20.0 μL of the 1000-ng/mL internal standard (short version of SD-101) aqueous solution was then added to all samples. Following vortex mixing for 1 minute, 100 μL of the 25/24/1 phenol/chloroform/isoamyl alcohol was added to each sample. The samples were then vortex-mixed using a Geno Grinder for 5 minutes at 1750 rpm and then centrifuged at 13,300 rpm for 15 minutes. The supernatants (250 μL) were transferred to a 96-well collection block, and the block was centrifuged at 3300 rpm for approximately 3 minutes.

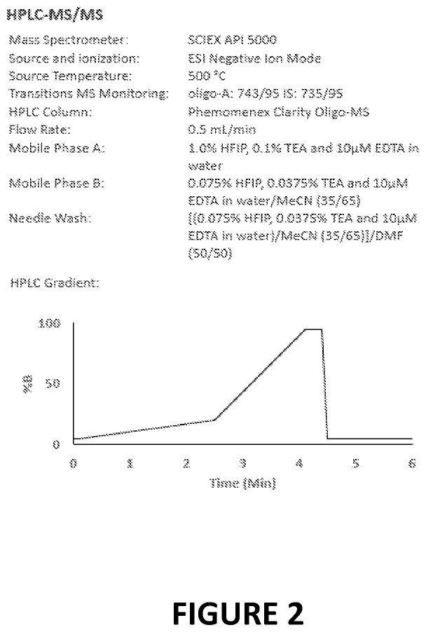

After extraction, samples were analyzed by HPLC-MS/MS as illustrated in FIG. 2. A Shimadzu HPLC system equipped with an autosampler and an AB Sciex API 5000 mass spectrometer was used for the LC-MS/MS analysis. Samples were injected on a Phenomenex Clarity Oligo-MS HPLC column. The mobile phases used were as follows:
Mobile Phase A: 1.0% HFIP, 0.1% TEA in water, 10 μM EDTA; and
Mobile Phase B: 0.075% HFIP, 0.0375% TEA in 65% MeCN and 35% water, 10 μM EDTA.

The needle wash was [(0.075% HFIP, 0.0375% TEA and 10 μM EDTA in water)/MeCN (35/65)]/DMF (50/50). Multiple reaction monitoring (MRM) was used to detect the analyte [precursor/product] transition (m/z) as follows: SD-101 [743/95] and SD-101 internal standard (SD-101 IS [735/95].

To determine the best homogenization buffer to use, SD-101 was spiked into control porcine plasma and control liver or lung homogenate with different homogenization buffer at QC-High level (350 ng/mL in plasma or tissue homogenate). The resulting samples were then extracted and analyzed. The 10 mM ammonium acetate in water, pH 4.5, showed the best result (see Table 1). Multiple tissue qualifications were performed with this homogenization buffer. When using the SD-101/SD-101 IS peak area ratio of porcine plasma sample as calibrates for the liver and lung QCs, accuracies of 93.6±9.0% and 97.3±1.7% were observed, respectively.

TABLE 1
Recovery ratio of porcine tissues exposed to test buffer solutions

| | Porcine Liver | Porcine Lung |
|---|---|---|
| | Homogenate | Homogenate |
| | (350 ng/mL | (350 ng/mL |
| Homogenization Buffer | SD-101) Recovery | SD-101) Recovery |
| Tris in H₂O, pH 8.0 | 86.8% ± 4.3% | 79.9% ± 3.2% |
| Tris in 20/80 methanol/H₂O, | 82.9% ± 2.2% | 86.0% ± 3.4% |
| pH 8.0* | | |
| Tris in 50/50 methanolH₂O, | 55.3% ± 3.2% | 87.6% ± 4.6% |
| pH 8.0 | | |
| Ammonium Acetate in H₂O, | 93.6% ± 9.0% | 97.3% ± 1.7% |
| pH 4.5 | | |
| Ammonium Acetate in 20/80 | 75.7% ± 2.8% | 97.9% ± 4.4% |
| MeOH/H₂O, pH 4.5 | | |
| Ammonium Acetate in 50/50 | 87.2% ± 4.5% | 55.6% ± 1.9% |
| MeOH/H₂O, pH 4.5 | | |

Next, with the selected homogenization buffer, the accuracy and precision qualification for this assay was performed. Each qualification run includes plasma calibration standards, plasma and tissue homogenate quality control samples. The acceptance criteria used for method qualification in this oligonucleotide tissue assay are as follows:

Calibration Standards: at least three-fourths (75%) must be within ±20.0% of nominal concentration (±25.0% for the LLOQ); and

QC Samples: at least two-thirds (67%) must be within ±20.0% of nominal concentration.

Further, at least one-half (50%) of the replicates at each QC concentration level must be within ±20.0% of the nominal concentration. Three analytical runs were performed to qualify the method for six different porcine tissues including liver, lung, brain, spleen, thymus and lymphoid tissue. Data from these analytical runs is summarized in Table 2.

TABLE 2
Accuracy and Precision (A&P) of plasma QC
samples in three tissue qualification runs

| | QC 1 | QC 2 | QC 3 | QC 4 |
|---|---|---|---|---|
| Plasma | 3.00 | 15.0 | 150 | 350 |
| Run 01 | 2.77 | 16.6 | 153 | 346 |
| Calculated Conc. | 2.84 | 13.7 | 146 | 362 |
| | 3.01 | 14.9 | 151 | 346 |
| Run 02 | 2.80 | 17.7 | 156 | 366 |
| Calculated Conc. | 2.70 | 15.7 | 151 | 366 |
| | 2.92 | 16.3 | 159 | 340 |
| Run 03 | 2.56 | 16.3 | 150 | 344 |
| Calculated Conc. | 3.25 | 13.9 | 144 | 352 |
| | 3.23 | 15.5 | 146 | 358 |
| Mean (ng/mL): | 2.90 | 15.6 | 151 | 353 |
| Std Dev (n = 9): | 0.232 | 1.29 | 4.90 | 9.95 |
| % CV: | 8.00 | 8.28 | 3.25 | 2.82 |
| % Difference: | −3.4 | 4.1 | 0.4 | 1.0 |

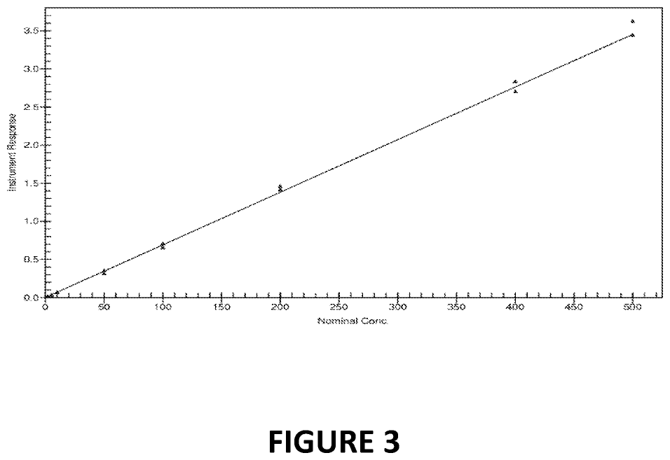

The data was generated using a linear calibration curve with 1/x² weighting for the determination of SD-101. This calibration curve was determined to be a best fit of the data. The representative porcine plasma calibration curve in tissue qualification is depicted in FIG. 3. The range of quantitation of the plasma curve was from 1.00 to 500 ng/mL (equivalent to 10.0 to 5000 ng/g for tissue). The coefficients of determination in the calibration standards for porcine plasma calibration standards ranged from 0.9836 to 0.9980. Based on the back-calculated concentrations using the regression curves, the percent difference of the mean calibration standards from the respective nominal concentrations ranged from −16.0% to 11.0%. Accuracy and precision were evaluated by replicate analyses of porcine plasma or tissue homogenate QC samples freshly prepared at concentrations of 3.00 ng/mL, 15.0 ng/mL, 150 ng/mL, and 350 ng/mL (equivalent to 30.0 ng/g, 150 ng/g, 1500 ng/g, and 3500 ng/g, respectively, in tissue). Precision was measured as the percent coefficient of variation (CV) of the concentrations at each level. Accuracy was expressed as the percent difference of the mean value for each pool from the nominal concentration. In plasma QCs, the percent difference from nominal concentration ranged from −17.0% to 10.0% and the precision (% CV) ranged from 2.0% to 9.0%. In tissue homogenate QCs, liver, lung, brain, spleen, thymus and lymphoid tissue QCs met the acceptance criteria. The percent difference from nominal concentration ranged from −16.7% to 14.0% and the precision (% CV) ranged from 0.39% to 17%. Table 3 summarizes data collected from samples quantified against the plasma curve.

TABLE 3
Accuracy and Precision of tissue QC samples
quantified against the plasma curve

| | QC 1 | QC 2 | QC 3 | QC 4 |
|---|---|---|---|---|
| | 3.00 | 15.0 | 150 | 350 |
| Liver | | | | |
| Calculated Conc. | 2.89 | 14.3 | 139 | 314 |
| | 3.25 | 13.9 | 127 | 330 |
| | 2.98 | 13.4 | 138 | 331 |
| Mean (ng/mL): | 3.04 | 13.9 | 135 | 325 |
| Std Dev (n = 3): | 0.187 | 0.451 | 6.66 | 9.54 |
| % CV: | 6.15 | 3.24 | 4.93 | 2.94 |
| % Difference: | 1.3 | −7.3 | −10.0 | −7.1 |
| Lung | | | | |
| Calculated Conc. | 2.88 | 14.3 | 148 | 338 |
| | 2.79 | 14.1 | 148 | 348 |
| | 2.89 | 16.0 | 147 | 361 |
| Mean (ng/mL): | 2.85 | 14.8 | 148 | 349 |
| Std Dev (n = 3): | 0.0551 | 1.04 | 0.577 | 11.5 |
| % CV: | 1.93 | 7.03 | 0.39 | 3.30 |
| % Difference: | −5.0 | −1.3 | −1.3 | −0.3 |
| Brain | | | | |
| Calculated Conc. | 2.64 | 12.3 | 132 | 320 |
| | 2.91 | 12.6 | 125 | 320 |
| | 2.56 | 12.6 | 132 | 309 |
| Mean (ng/mL): | 2.70 | 12.5 | 130 | 316 |
| Std Dev (n = 3): | 0.183 | 0.173 | 4.04 | 6.35 |
| % CV: | 6.78 | 1.38 | 3.11 | 2.01 |
| % Difference: | −10.0 | −16.7 | −13.3 | −9.7 |
| Spleen | | | | |
| Calculated Conc. | 2.93 | 13.1 | 129 | 306 |
| | 2.43 | 13.5 | 143 | 318 |
| | 2.66 | 12.2 | 126 | 307 |
| Mean (ng/mL): | 2.67 | 12.9 | 133 | 310 |
| Std Dev (n = 3): | 0.250 | 0.666 | 9.07 | 6.66 |
| % CV: | 9.36 | 5.16 | 6.82 | 2.15 |
| % Difference: | −11.0 | −14.0 | −11.3 | −11.4 |
| Thymus | | | | |
| Calculated Conc. | 2.45 | 13.1 | 133 | 320 |
| | 2.47 | 12.8 | 126 | 309 |
| | 3.11 | 12.4 | 126 | 308 |
| Mean (ng/mL): | 2.68 | 12.8 | 128 | 312 |
| Std Dev (n = 3): | 0.375 | 0.351 | 4.04 | 6.66 |
| % CV: | 14.0 | 2.74 | 3.16 | 2.13 |
| % Difference: | −10.7 | −14.7 | −14.7 | −10.9 |
| Run | | | | |
| Calculated Conc. | 3.21 | 12.0 | 136 | 307 |
| | 2.48 | 12.9 | 125 | 327 |
| | 2.70 | 13.6 | 133 | 308 |
| Mean (ng/mL): | 2.80 | 12.8 | 131 | 314 |
| Std Dev (n = 3): | 0.374 | 0.802 | 5.69 | 11.3 |
| % CV: | 13.4 | 6.27 | 4.34 | 3.60 |
| % Difference: | −6.7 | −14.7 | −12.7 | −10.3 |

Further to the above, after the selection of the homogenization buffer corresponding to 10 mM ammonium acetate in water, with a pH 4.5, a range of buffer-to-tissue ratios were evaluated (e.g., 4:1 to 200:1) with and without the addition of proteinase K (e.g., 50 μg to 200 μg of proteinase K for every 1 mL of buffer).

In sum, the exemplary method can be used to quantitate SD-101 in six tissues with a calibration curve made with plasma. This method can be used to determine the tissue distribution of SD-101. Moreover, the utilization of a matrix/buffer matching for various tissue samples and plasma calibration curve allows for the use of one analytical method for the quantitation of an oligonucleotide in early-stage development.

Further, although the exemplary method evaluated porcine tissue, human tissue can be similarly used.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method for quantitation of an oligonucleotide within a tissue, the method comprising:
mixing the tissue with a homogenization buffer composition to create a tissue homogenate, wherein the homogenization buffer composition comprises ammonium acetate in solution in water;
adding the tissue homogenate to a quantity of oligonucleotide-free plasma to create a tissue homogenate solution;
adding a quantity of a reference standard oligonucleotide to the tissue homogenate solution to create a homogenate/plasma/standard solution;
adding a quantity of a phenol/chloroform/isoamyl alcohol to the homogenate/plasma/standard solution;
employing centrifugal force to separate a supernatant from the homogenate/plasma/standard solution; and
analyzing the supernatant for a concentration of the oligonucleotide, wherein the oligonucleotide is a toll-like receptor 9 agonist oligonucleotide having the structure: 5′-TCG AAC GTT CGA ACG TTC GAA CGT TCG AAT-3′ (SEQ ID NO: 1).

2. The method of claim 1, wherein a concentration of the ammonium acetate within the homogenization buffer composition is about 5 mM to 20 mM.

3. The method of claim 2, wherein the concentration of the ammonium acetate within the homogenization buffer composition is 10 mM.

4. The method of claim 1, wherein the pH of the homogenization buffer composition is between 3.0 to 6.0 pH.

5. The method of claim 4, wherein the pH of the homogenization buffer composition is 4.5 pH.

6. The method of claim 1, wherein the homogenization buffer composition is in a concentration ranging from 4 mL of the homogenization buffer composition per 1 gram of the tissue to 200 mL of the homogenization buffer composition per 1 gram of the tissue.

7. The method of claim 6, wherein the homogenization buffer composition is in a concentration ranging from 20 mL of the homogenization buffer composition per 1 gram of the tissue to 100 mL of the homogenization buffer composition per 1 gram of the tissue.

8. The method of claim 7, wherein the homogenization buffer composition is in a concentration ranging from 20 mL of the homogenization buffer composition per 1 gram of the tissue to 50 mL of the homogenization buffer composition per 1 gram of the tissue.

9. The method of claim 8, wherein the homogenization buffer composition is in a concentration of 50 mL of the homogenization buffer composition per 1 gram of the tissue.

10. The method of claim 1, wherein the homogenization buffer composition further comprises a quantity of proteinase K.

11. The method of claim 10, wherein the quantity of proteinase K is from 50 μg to 200 μg for every 1 mL of the homogenization buffer composition.

12. The method of claim 11, wherein the quantity of proteinase K is 50 μg for every 1 mL of the homogenization buffer composition.

13. The method of claim 1, wherein the tissue homogenate solution comprises oligonucleotide-free plasma and tissue homogenate at a 50/50 volume ratio.

14. The method of claim 1, wherein the homogenate/plasma/standard solution comprises 200 μL of tissue homogenate, 200 μL of oligonucleotide-free plasma, and 20.0 μL of reference standard oligonucleotide.

15. The method of claim 1, wherein the quantity of the phenol/chloroform/isoamyl alcohol is 100 μL.

16. The method of claim 1, wherein the phenol/chloroform/isoamyl alcohol is 25/24/1 phenol/chloroform/isoamyl alcohol v/v.

17. The method of claim 1, wherein the supernatant is analyzed for the concentration of the oligonucleotide by high performance liquid chromatography tandem mass spectroscopy (HPLC-MS/MS).

18. The method of claim 1, wherein the tissue is any one of liver, lung, brain, spleen, thymus, and lymphoid tissue.

19. The method of claim 18, wherein the tissue does not include any cancerous tumors.

20. A homogenization buffer composition for quantitation of an oligonucleotide within a tissue, the homogenization buffer composition comprising:
ammonium acetate in solution in water, wherein a concentration of the ammonium acetate within the homogenization buffer composition is about 5 mM to 20 mM; and
a quantity of proteinase K, wherein the quantity of proteinase K is from 50 μg to 200 μg for every 1 mL of the homogenization buffer composition;
wherein:
the pH of the homogenization buffer composition is between 3.0 to 6.0 pH; and
the homogenization buffer composition is in a concentration ranging from 4 mL of the homogenization buffer composition per 1 gram of the tissue to 200 mL of the homogenization buffer composition per 1 gram of the tissue.
